(19) United States
(12) Patent Application Publication
(10) Pub. No.: US 2012/0123374 A1
(43) Pub. Date: May 17, 2012

(54) Meltblown Nonwoven Compositions and Methods for Making Them

(76) Inventors: Galen C. Richeson, Humble, TX (US); Alistair D. Westwood, Kingwood, TX (US)

(21) Appl. No.: 13/276,035

(22) Filed: Oct. 18, 2011

Related U.S. Application Data
(60) Provisional application No. 61/411,708, filed on Nov. 9, 2010.

(51) Int. Cl. A61L 15/24; D04H 13/00

(52) U.S. Cl. 604/372; 442/328

(57) ABSTRACT

Meltblown nonwoven compositions and processes for forming them are described herein. In one or more embodiments, the invention is directed to meltblown nonwoven fabrics having at least one elastic layer, wherein the elastic layer comprises a propylene-based polymer having an MFR greater than about 25 g/10 min. Additionally, the propylene-based polymer comprises from about 5 to about 25 wt % of one or more C₂ and/or C₄-C₁₂ α-olefins and has a triad tacticity greater than about 90% and a heat of fusion less than about 75 J/g. The present invention is also directed to processes for forming meltblown nonwoven fabrics comprising forming a molten propylene-based polymer having an MFR of at least about 25 g/10 min, forming fibers comprising the propylene-based polymer, and forming an elastic nonwoven layer from the fibers.

### PRIORITY CLAIM

This application claims priority to U.S. Ser. No. 61/411,708, filed Nov. 9, 2010, the disclosure of which is herein incorporated by reference in its entirety.

### CROSS REFERENCE TO RELATED APPLICATIONS

The present application relates to U.S. Ser. No. 12/271,526, filed Nov. 14, 2008 (2008EM290), U.S. Ser. No. 61/101,341, filed Sep. 30, 2008 (2008EM066), U.S. Ser. No. 61/157,524, filed Mar. 24, 2009 (2008EM066A), U.S. Ser. No. 12/566,564, filed Sep. 24, 2009 (2008EM066A/2), U.S. Ser. No. 61/156,078, filed Feb. 27, 2009 (2008EM066B), U.S. Ser. No. 12/566,410, filed Sep. 24, 2009 (2008EM066B/2), U.S. Ser. No. 61/171,135, filed Apr. 21, 2009 (2008EM066C), U.S. Ser. No. 12/566,434, filed Sep. 24, 2009 (2008EM066C/2), U.S. Ser. No. 61/248,254, filed Oct. 2, 2009 (2009EM208), U.S. Ser. No. 12/894,955, filed Sep. 30, 2010 (2008EM208/2), U.S. Ser. No. 12/723,317, filed Mar. 12, 2010 (2010EM070), U.S. Ser. No. 12/723,336, filed Mar. 12, 2010 (2010EM071), and U.S. Ser. No. 12/726,642, filed Mar. 18, 2010 (2010EM091), each of which are herein incorporated by reference in their entireties.

### BACKGROUND OF THE INVENTION

Propylene-based polymers and copolymers are well known in the art for their usefulness in the manufacture of meltblown nonwoven fabrics. Such fabrics have a wide variety of uses, such as in medical and hygiene products, clothing, filter media, and sorbent products. Meltblown nonwoven fabrics are particularly useful in hygiene products, such as baby diapers, adult incontinence products, and feminine hygiene products. An important aspect of these fabrics, particularly in hygiene applications, is the ability to produce aesthetically pleasing fabrics having good leakage performance at a low cost. Good leakage performance is achieved via the elasticity of the elastic layers of the fabrics, which provides better fit and conformity to the wearer, resulting in fewer leaks.

Production of meltblown nonwoven fabrics using commercially available propylene-based polymers having a melt flow rate (MFR) less than 25 g/10 min in the elastic layers can be difficult, because the low MFR of such polymers requires high melt temperatures and high pressures to melt blow. High process temperatures can cause undesirable degradation in the extruder, while high pressures limit the throughput rate of the melt blowing equipment. Previously, some propylene-based polymers have been blended with an additional polymer or polymers post-reactor, often isotactic propylene homopolymers, and then visbroken with peroxide to achieve a higher MFR polymer for use in elastic layers. Blending with an additional polymer is undesirable as well, however, because it increases manufacturing cost and reduces the elastic performance of the resulting nonwoven fabrics.

It is therefore desirable to form the elastic layers of meltblown nonwoven fabrics from propylene-based polymers having a higher MFR (i.e., greater than 25 g/10 min) without blending the propylene-based polymers post-reactor with homopolypropylene or other polymers. Such fabrics can be produced under broader process conditions, and at higher throughput rates and lower costs.

United States Patent Application Publication No. 2002/0019507 describes propylene-based polymers that have been visbroken with peroxide to increase the MFR of the polymer for use in adhesive applications.

United States Patent Application Publication No. 2005/0130544 describes blends of propylene-based polymers with propylene homopolymers for use in fiber spinning applications.

United States Patent Application Publication No. 2008/0172840 describes polyolefin blend compositions suitable for use in spunbond fiber or filament applications having an MFR between 100 and 500 g/10 min.

United States Patent Application Publication No. 2009/0124154 describes nonwoven fabrics comprising two or more propylene-based elastomers and one or more propylene-based thermoplastic polymers.

International Publication No. WO2009/064583 describes nonwoven fabrics made from compositions comprising a low crystallinity propylene-based elastomer, a high crystallinity propylene-based elastomer, and a propylene-based thermoplastic polymer.

### BRIEF DESCRIPTION OF THE FIGURES

FIGS. 1 and 2 show die pressure versus throughput rate for the meltblown extrusion of a comparative polymer and for polymers of the invention.

### SUMMARY OF THE INVENTION

The present invention is directed to meltblown nonwoven compositions, and to processes for forming them. In one or more embodiments, the invention is directed to meltblown nonwoven fabrics having at least one elastic layer, wherein the elastic layer comprises a propylene-based polymer having an MFR greater than about 25 g/10 min. Additionally, the propylene-based polymer comprises from about 5 to about 25 wt % of one or more C₂ and/or C₄-C₁₂ α-olefins and has a triad tacticity greater than about 90% and a heat of fusion less than about 75 J/g. The propylene-based polymer may be a reactor grade polymer having an MFR greater than about 25 g/10 min, or the propylene-based polymer may be a lower MFR polymer that is visbroken with peroxide or another prodegradant to increase the MFR. In some embodiments, the elastic layer comprises a visbroken propylene-based polymer, but does not include additional polymers. In particular embodiments, the elastic layer is substantially free of additional homopolymers or copolymers of propylene added post-reactor. The present invention is also directed to processes for forming meltblown nonwoven fabrics comprising forming a molten propylene-based polymer having an MFR of at least about 25 g/10 min, forming fibers comprising the propylene-based polymer, and forming an elastic nonwoven layer from the fibers.

### DETAILED DESCRIPTION OF THE INVENTION

The present invention is directed to meltblown nonwoven fabrics having at least one elastic layer, wherein the elastic layer comprises a propylene-based polymer, and to processes for forming such fabrics. In certain embodiments, the propylene-based polymer comprises propylene and from about 5 to about 25 wt % units derived from ethylene and/or a C₄-C₁₀ α-olefin. The propylene-based copolymer also has a melt flow rate (MFR) greater than about 25 g/10 min, a heat of fusion (Hf) less than about 75 J/g, and a triad tacticity greater than about 90%.

As used herein, the term “copolymer” is meant to include polymers having two or more monomers, optionally with other monomers, and may refer to interpolymers, terpolymers, etc. The term “polymer” as used herein includes, but is not limited to, homopolymers, copolymers, terpolymers, etc., and alloys and blends thereof. The term “polymer” as used herein also includes impact, block, graft, random and alternating copolymers. The term “polymer” shall further include all possible geometrical configurations unless otherwise specifically stated. Such configurations may include isotactic, syndiotactic and random symmetries. The term “blend” as used herein refers to a mixture of two or more polymers.

The term “monomer” or “comonomer” as used herein can refer to the monomer used to form the polymer, i.e., the unreacted chemical compound in the form prior to polymerization, and can also refer to the monomer after it has been incorporated into the polymer, also referred to herein as a “[monomer]-derived unit”, which by virtue of the polymerization reaction typically has fewer hydrogen atoms than it does prior to the polymerization reaction. Different monomers are discussed herein, including propylene monomers, ethylene monomers, and diene monomers.

“Polypropylene” as used herein includes homopolymers and copolymers of propylene or mixtures thereof. Products that include one or more propylene monomers polymerized with one or more additional monomers may be more commonly known as random copolymers (RCP) or impact copolymers (ICP). Impact copolymers are also known in the art as heterophasic copolymers. “Propylene-based,” as used herein, is meant to include any polymer comprising propylene, either alone or in combination with one or more comonomers, in which propylene is the major component (i.e., greater than 50 wt % propylene).

“Reactor grade” as used herein means a polymer that has not been chemically or mechanically treated or blended after polymerization in an effort to alter the polymer's average molecular weight, molecular weight distribution, or viscosity. Particularly excluded from those polymers described as reactor grade are those that have been visbroken or otherwise treated or coated with peroxide. For the purposes of this disclosure, however, reactor grade polymers do include those polymers that are reactor blended, i.e., where two or more polymers are formed in situ in a single reactor, a series of reactors, or parallel reactors resulting in a polymer blend.

“Visbreaking” as used herein is a process for reducing the molecular weight of a polymer by subjecting the polymer to chain scission. The visbreaking process also increases the MFR of a polymer and may narrow its molecular weight distribution. Several different types of chemical reactions can be employed for visbreaking propylene-based polymers. An example is thermal pyrolysis, which is accomplished by exposing a polymer to high temperatures, e.g., in an extruder at 350° C. or higher. Other approaches are exposure to powerful oxidizing agents and exposure to ionizing radiation. The most commonly used method of visbreaking in commercial practice is the addition of a prodegradant to the polymer. A prodegradant is a substance that promotes chain scission when mixed with a polymer, which is then heated under extrusion conditions. Examples of prodegradants used in commercial practice are alkyl hydroperoxides and dialkyl peroxides. These materials, at elevated temperatures, initiate a free radical chain reaction resulting in scission of polypropylene molecules. The terms “prodegradant” and “visbreaking agent” are used interchangeably herein. Polymers that have undergone chain scission via a visbreaking process are said herein to be “visbroken.” Such visbroken polymer grades, particularly polypropylene grades, are often referred to in the industry as “controlled rheology” or “CR” grades.

### Propylene-Based Polymers

The meltblown nonwoven fabrics of the present invention comprise one or more elastic layers. In some embodiments, the one or more elastic layers comprise a propylene-based polymer, which comprises propylene and from about 5 to about 25 wt % of one or more comonomers selected from ethylene and/or C₄-C₁₂ α-olefins. In one or more embodiments, the α-olefin comonomer units may derive from ethylene, butene, pentene, hexene, 4-methyl-1-pentene, octene, or decene. The embodiments described below are discussed with reference to ethylene as the α-olefin comonomer, but the embodiments are equally applicable to other copolymers with other α-olefin comonomers. In this regard, the copolymer may simply be referred to as propylene-based polymers with reference to ethylene as the α-olefin.

In one or more embodiments, the propylene-based polymer may include at least about 5 wt %, at least about 6 wt %, at least about 7 wt %, or at least about 8 wt %, or at least about 10 wt %, or at least about 12 wt % ethylene-derived units. In those or other embodiments, the copolymers may include up to about 25 wt %, or up to about 22 wt %, or up to about 20 wt %, or up to about 19 wt %, or up to about 18 wt %, or up to about 17 wt % ethylene-derived units, where the percentage by weight is based upon the total weight of the propylene-derived and α-olefin derived units. Stated another way, the propylene-based polymer may include at least about 75 wt %, or at least about 80 wt %, or at least about 81 wt % propylene-derived units, or at least about 82 wt % propylene-derived units, or at least about 83 wt % propylene-derived units; and in these or other embodiments, the copolymers may include up to about 95 wt %, or up to about 94 wt %, or up to about 93 wt %, or up to about 92 wt %, or up to about 90 wt %, or up to about 88 wt % propylene-derived units, where the percentage by weight is based upon the total weight of the propylene-derived and alpha-olefin derived units.

The propylene-based polymers of one or more embodiments are characterized by a melting point (Tm), which can be determined by differential scanning calorimetry (DSC). For purposes herein, the maximum of the highest temperature peak is considered to be the melting point of the polymer. A “peak” in this context is defined as a change in the general slope of the DSC curve (heat flow versus temperature) from positive to negative, forming a maximum without a shift in the baseline where the DSC curve is plotted so that an endothermic reaction would be shown with a positive peak.

In one or more embodiments, the Tm of the propylene-based polymer (as determined by DSC) is less than about 115° C., or less than about 110° C., or less than about 100° C., or less than about 90° C.

In one or more embodiments, the propylene-based polymer may be characterized by its heat of fusion (Hf), as determined by DSC. In one or more embodiments, the propylene-based polymer may have an Hf that is at least about 0.5 J/g, or at least about 1.0 J/g, or at least about 1.5 J/g, or at least about 3.0 J/g, or at least about 4.0 J/g, or at least about 6.0 J/g, or at least about 7.0 J/g. In these or other embodiments, the propylene-based copolymer may be characterized by an Hf of less than about 75 J/g, or less than about 70 J/g, or less than about 60 J/g, or less than about 50 J/g, or less than about 30 J/g.

As used within this specification, DSC procedures for determining Tm and Hf include the following. The polymer is pressed at a temperature of from about 200° C. to about 230° C. in a heated press, and the resulting polymer sheet is hung, under ambient conditions, in the air to cool. About 6 to 10 mg of the polymer sheet is removed with a punch die. This 6 to 10 mg sample is annealed at room temperature for about 80 to 100 hours. At the end of this period, the sample is placed in a DSC (Perkin Elmer Pyris One Thermal Analysis System) and cooled to about −50° C. to about −70° C. The sample is heated at 10° C./min to attain a final temperature of about 200° C. The sample is kept at 200° C. for 5 minutes and a second cool-heat cycle is performed. Events from both cycles are recorded. The thermal output is recorded as the area under the melting peak of the sample, which typically occurs between about 0° C. and about 200° C. It is measured in Joules and is a measure of the Hf of the polymer.

The propylene-based polymer can have a triad tacticity of three propylene units, as measured by ¹³C NMR, of 75% or greater, 80% or greater, 85% or greater, 90% or greater, 92% or greater, 95% or greater, or 97% or greater. In one or more embodiments, ranges include from about 75 to about 99%, in other embodiments from about 80 to about 99%, in other embodiments from about 85 to about 99%, in other embodiments from about 90 to about 99%, and in other embodiments from about 80 to about 97%. Triad tacticity is determined by the methods described in U.S. Patent Application Publication No. 2004/0236042.

The propylene-based polymer may have a tacticity index m/r ranging from a lower limit of 4 or 6 to an upper limit of 8 or 10 or 12. The tacticity index, expressed herein as “m/r”, is determined by ¹³C nuclear magnetic resonance (“NMR”). The tacticity index m/r is calculated as defined by H. N. Cheng in 17 MACROMOLECULES, pp. 1950-1955 (1984), incorporated herein by reference. The designation “m” or “r” describes the stereochemistry of pairs of contiguous propylene groups, “m” referring to meso and “r” to racemic. An m/r ratio of 1.0 generally describes a syndiotactic polymer, and an m/r ratio of 2.0 an atactic material. An isotactic material theoretically may have a ratio approaching infinity, and many by-product atactic polymers have sufficient isotactic content to result in ratios of greater than 50.

In one or more embodiments, the propylene-based polymer may have a percent crystallinity of from about 0.5% to about 40%, or from about 1% to about 30%, or from about 5% to about 25%, determined according to DSC procedures. Crystallinity may be determined by dividing the H_{f} of a sample by the H_{f} of a 100% crystalline polymer, which is assumed to be 189 joules/gram for isotactic polypropylene or 350 joules/gram for polyethylene.

In one or more embodiments, the propylene-based polymer may have a density of from about 0.85 g/cm³ to about 0.92 g/cm³, or from about 0.86 g/cm³ to about 0.90 g/cm³, or from about 0.86 g/cm³ to about 0.89 g/cm³ at room temperature as measured per the ASTM D-792 test method.

In one or more embodiments, the propylene-based polymer can have a melt index (MI) (ASTM D-1238, 2.16 kg @ 190° C.), of greater than or equal to about 10 g/10 min, or greater than or equal to about 20 g/10 min, or greater than or equal to about 25 g/10 min, or greater than or equal to about 30 g/10 min.

In one or more embodiments, the propylene-based polymer can have a melt flow rate (MFR), as measured according to ASTM D-1238, 2.16 kg weight @ 230° C., greater than about 25 g/10 min, or greater than about 30 g/10 min, or greater than about 40 g/10 min, or greater than about 50 g/10 min, or greater than about 60 g/10 min, or greater than about 70 g/10 min, or greater than about 80 g/10 min, or greater than about 90 g/10 min. In some embodiments, the propylene-based polymer is a reactor grade polymer having an MFR greater than about 25 g/10 min. In other words, the polymer has an MFR greater than 25 g/10 min as it is made in the reactor, and it is not treated or blended with another polymer to increase the MFR after it exits the reactor.

In other embodiments, the propylene-based polymer may be a polymer that has an MFR lower than 25 g/10 min as it is made in the reactor that is visbroken after exiting the reactor to increase the MFR. In one or more embodiments, the propylene-based polymer is treated with a visbreaking agent such that the melt flow rate of the polymer after treatment is at least 1.5 times the initial MFR of the polymer prior to visbreaking. Alternately, the propylene-based polymer may be treated with a visbreaking agent such that the MFR is increased by 2 times, or 2.5 times, or 3 times, or 3.5 times, or 4 times the MFR of the polymer prior to visbreaking.

In the same or other embodiments, the propylene-based polymer may be treated with from about 100 to about 5000 parts per million (ppm), or from about 150 to about 4000 ppm, or from about 200 to about 3000 ppm, or from about 250 to about 2500 ppm, or from about 250 to about 2000 ppm, of a visbreaking agent in order to achieve the desired final MFR of the polymer. In one or more embodiments, the visbreaking agent employed is an organic peroxide. Suitable organic peroxides include, but are not limited to, dialkyl peroxides, diacyl peroxides, hydroperoxides, cyclic peroxides, peroxyesters, and peroxyketals. In particular, preferred peroxides include 2,5-dimethyl-2,5-di(t-butylperoxy)hexane, 2,5-dimethyl-2,5-di(t-butylperoxy)hexyne-3, di-(t-butyl)peroxide, di-(t-butylperoxyisopropyl)benzene, 3,6,9-triethyl-3,6,9-trimethyl-1,4,7-triperoxonane, dicumyl peroxide, and di-(t-amyl)peroxide. Such peroxides are commercially available under the tradenames Di-Cup, Vul-Cup, and Luperox from Arkema, Inc., and under the tradenames Trigonox and Perkadox from Akzo Nobel, Inc. In one or more embodiments of the invention, the propylene-based polymer is treated with from about 250 to about 2500 ppm of 2,5-dimethyl-2,5-di(t-butylperoxy)hexane such that the MFR of the polymer after treatment is at least 25 g/10 min, or at least 30 g/10 min, or at least 1.5 times the original MFR of the polymer.

In one or more embodiments, the propylene-based polymer may have a Mooney viscosity [ML (1+4)@ 125° C.], as determined according to ASTM D-1646, of less than about 100, or less than about 75, or less than about 50, or less than about 30.

In one or more embodiments, the propylene-based polymer may have a g′ index value of 0.95 or greater, or at least 0.97, or at least 0.99, wherein g′ is measured at the Mw of the polymer using the intrinsic viscosity of isotactic polypropylene as the baseline. For use herein, the g′ index is defined as:

$${g}^{′} = \frac{{η}_{b}}{{η}_{l}}$$

where θ_{b} is the intrinsic viscosity of the polymer and ηₗ is the intrinsic viscosity of a linear polymer of the same viscosity-averaged molecular weight (Mᵥ) as the polymer. ηₗ=KMᵥ^{α}, K and α are measured values for linear polymers and should be obtained on the same instrument as the one used for the g′ index measurement.

In one or more embodiments, the propylene-based polymer can have a weight average molecular weight (Mw) of from about 5,000 to about 1,000,000 g/mole, or from about 10,000 to about 500,000 g/mole, or from about 20,000 to about 400,000 g/mole, or from about 50,000 to about 300,000 g/mole.

In one or more embodiments, the propylene-based polymer can have a number average molecular weight (Mn) of from about 2,500 to about 2,500,000 g/mole, or from about 5,000 to about 500,000 g/mole, or from about 10,000 to about 250,000 g/mole, or from about 25,000 to about 200,000 g/mole.

In one or more embodiments, the propylene-based polymer can have a Z-average molecular weight (Mz) of from about 10,000 to about 7,000,000 g/mole, or from about 50,000 to about 1,000,000 g/mole, or from about 80,000 to about 700,000 g/mole, or from about 100,000 to about 500,000 g/mole.

In one or more embodiments, the molecular weight distribution (MWD=(Mw/Mn)) of the propylene-based polymer may be from about 1 to about 40, or from about 1 to about 15, or from about 1.8 to about 5, or from about 1.8 to about 3.

Techniques for determining the molecular weight (Mn, Mw and Mz) and MWD may be found in U.S. Pat. No. 4,540,753 (Cozewith, Ju and Ver Strate) (which is incorporated by reference herein for purposes of U.S. practices) and references cited therein and in Macromolecules, 1988, Vol. 21, p. 3360 (Ver Strate et al.), which is herein incorporated by reference for purposes of U.S. practices, and references cited therein. For example, molecular weight may be determined by size exclusion chromatography (SEC) by using a Waters 150 gel permeation chromatograph equipped with the differential refractive index detector and calibrated using polystyrene standards.

Optionally, the propylene-based polymer may also include one or more dienes. The term “diene” is defined as a hydrocarbon compound that has two unsaturation sites, i.e., a compound having two double bonds connecting carbon atoms. Depending on the context, the term “diene” in this patent refers broadly to either a diene monomer prior to polymerization, e.g., forming part of the polymerization medium, or a diene monomer after polymerization has begun (also referred to as a diene monomer unit or a diene-derived unit). Exemplary dienes suitable for use in the present invention include, but are not limited to, butadiene, pentadiene, hexadiene (e.g., 1,4-hexadiene), heptadiene (e.g., 1,6-heptadiene), octadiene (e.g., 1,7-octadiene), nonadiene (e.g., 1,8-nonadiene), decadiene (e.g., 1,9-decadiene), undecadiene (e.g., 1,10-undecadiene), dodecadiene (e.g., 1,11-dodecadiene), tridecadiene (e.g., 1,12-tridecadiene), tetradecadiene (e.g., 1,13-tetradecadiene), pentadecadiene, hexadecadiene, heptadecadiene, octadecadiene, nonadecadiene, icosadiene, heneicosadiene, docosadiene, tricosadiene, tetracosadiene, pentacosadiene, hexacosadiene, heptacosadiene, octacosadiene, nonacosadiene, triacontadiene, and polybutadienes having a molecular weight (Mw) of less than 1000 g/mol. Examples of straight chain acyclic dienes include, but are not limited to 1,4-hexadiene and 1,6-octadiene. Examples of branched chain acyclic dienes include, but are not limited to 5-methyl-1,4-hexadiene, 3,7-dimethyl-1,6-octadiene, and 3,7-dimethyl-1,7-octadiene. Examples of single ring alicyclic dienes include, but are not limited to 1,4-cyclohexadiene, 1,5-cyclooctadiene, and 1,7-cyclododecadiene. Examples of multi-ring alicyclic fused and bridged ring dienes include, but are not limited to tetrahydroindene; norbornadiene; methyltetrahydroindene; dicyclopentadiene; bicyclo(2.2.1)hepta-2,5-diene; and alkenyl-, alkylidene-, cycloalkenyl-, and cylcoalkylidene norbornenes [including, e.g., 5-methylene-2-norbornene, 5-ethylidene-2-norbornene, 5-propenyl-2-norbornene, 5-isopropylidene-2-norbornene, 5-(4-cyclopentenyl)-2-norbornene, 5-cyclohexylidene-2-norbornene, and 5-vinyl-2-norbornene]. Examples of cycloalkenyl-substituted alkenes include, but are not limited to vinyl cyclohexene, allyl cyclohexene, vinylcyclooctene, 4-vinylcyclohexene, allyl cyclodecene, vinylcyclododecene, and tetracyclododecadiene. In some embodiments of the present invention, the diene is selected from 5-ethylidene-2-norbornene (ENB); 1,4-hexadiene; 5-methylene-2-norbornene (MNB); 1,6-octadiene; 5-methyl-1,4-hexadiene; 3,7-dimethyl-1,6-octadiene; 1,3-cyclopentadiene; 1,4-cyclohexadiene; vinyl norbornene (VNB); dicyclopentadiene (DCPD), and combinations thereof. In one or more embodiments, the diene is ENB.

In some embodiments, the propylene-based polymer comprises from 0.05 to about 6 wt % diene-derived units. In further embodiments, the polymer comprises from about 0.1 to about 5.0 wt % diene-derived units, or from about 0.25 to about 3.0 wt % diene-derived units, or from about 0.5 to about 1.5 wt % diene-derived units.

### Preparation of the Propylene-Based Polymers

Polymerization of the propylene-based polymer is conducted by reacting monomers in the presence of a catalyst system described herein at a temperature of from 0° C. to 200° C. for a time of from 1 second to 10 hours. Preferably homogeneous conditions are used, such as a continuous solution process or a bulk polymerization process with excess monomer used as diluent. The continuous process may use some form of agitation to reduce concentration differences in the reactor and maintain steady state polymerization conditions. The heat of the polymerization reaction is preferably removed by cooling of the polymerization feed and allowing the polymerization to heat up to the polymerization, although internal cooling systems may be used.

The polymers of the invention can be prepared with the catalysts described in either batch, semi-continuous, or continuous propylene polymerization systems. Desirable polymerization systems are the continuous processes, including diluent slurry, bulk slurry (loop and stirred tank), and gas phase (stirred and fluid bed). Continuous polymerization can be carried out in a single reactor of any of the above types, in two or more reactors operating in series, or in two or more reactors operating in parallel. When two or more reactors are operating in a continuous process, the multiple reactors can be all of the same type or they may be any combination of the types.

Further description of exemplary methods suitable for preparation of the propylene-based polymers described herein may be found in U.S. Pat. No. 6,881,800, which is incorporated by reference herein for purposes of U.S. practice.

In one or more embodiments herein, the propylene-based polymers may be part of a reactor blend comprising one or more additional polymers. The polymer blend may be produced by any reactor blend method currently known in the art. A reactor blend is a highly dispersed and mechanically inseparable blend of the polymers produced in situ as the result of sequential polymerization of one or more monomers with the formation of one polymer in the presence of another. The polymers may be produced in any of the polymerization methods described above. The reactor blends are produced in a single reactor, a series of reactors, or parallel reactors and are reactor grade blends. In other words, substantially no additional homopolymers or copolymers of propylene are added to the blends after exiting the reactor.

The triad tacticity and tacticity index of the propylene-based copolymer may be controlled by the catalyst, which influences the stereoregularity of propylene placement, the polymerization temperature, according to which stereoregularity can be reduced by increasing the temperature, and by the type and amount of a comonomer, which tends to reduce the level of longer propylene derived sequences.

Too much comonomer may reduce the crystallinity provided by the crystallization of stereoregular propylene derived sequences to the point where the material lacks strength; too little and the material may be too crystalline. The comonomer content and sequence distribution of the polymers can be measured using ¹³C nuclear magnetic resonance (NMR) by methods well known to those skilled in the art. Comonomer content of discrete molecular weight ranges can be measured using methods well known to those skilled in the art, including Fourier Transform Infrared Spectroscopy (FTIR) in conjunction with samples by GPC, as described in Wheeler and Willis, Applied Spectroscopy, 1993, Vol. 47, pp. 1128-1130. For a propylene ethylene copolymer containing greater than 75 wt % propylene, the comonomer content (ethylene content) of such a polymer can be measured as follows: A thin homogeneous film is pressed at a temperature of about 150° C. or greater, and mounted on a Perkin Elmer PE 1760 infrared spectrophotometer. A full spectrum of the sample from 600 cm-1 to 4000 cm-1 is recorded and the monomer weight percent of ethylene can be calculated according to the following equation: Ethylene wt %=82.585−111.987X+30.045X2, where X is the ratio of the peak height at 1155 cm-1 and peak height at either 722 cm-1 or 732 cm-1, whichever is higher. For propylene ethylene copolymers having 75 wt % or less propylene content, the comonomer (ethylene) content can be measured using the procedure described in Wheeler and Willis.

Reference is made to U.S. Pat. No. 6,525,157, whose test methods are also fully applicable for the various measurements referred to in this specification and claims and which contains more details on GPC measurements, the determination of ethylene content by NMR and the DSC measurements.

The catalyst may also control the stereoregularity in combination with the comonomer and the polymerization temperature. The propylene-based polymers described herein are prepared using one or more catalyst systems. As used herein, a “catalyst system” comprises at least a transition metal compound, also referred to as catalyst precursor, and an activator. Contacting the transition metal compound (catalyst precursor) and the activator in solution upstream of the polymerization reactor or in the polymerization reactor of the disclosed processes yields the catalytically active component (catalyst) of the catalyst system. Any given transition metal compound or catalyst precursor can yield a catalytically active component (catalyst) with various activators, affording a wide array of catalysts deployable in the processes of the present invention. Catalyst systems of the present invention comprise at least one transition metal compound and at least one activator. However, catalyst systems of the current disclosure may also comprise more than one transition metal compound in combination with one or more activators. Such catalyst systems may optionally include impurity scavengers. Each of these components is described in further detail below.

In one or more embodiments of the present invention, the catalyst systems used for producing propylene-based polymers comprise a metallocene compound. In some embodiments, the metallocene compound is a bridged bisindenyl metallocene having the general formula (In¹)Y(In²)MX₂, where In¹ and In² are identical substituted or unsubstituted indenyl groups bound to M and bridged by Y, Y is a bridging group in which the number of atoms in the direct chain connecting In¹ with In² is from 1 to 8 and the direct chain comprises C or Si, and M is a Group 3, 4, 5, or 6 transition metal. In¹ and In² may be substituted or unsubstituted. If In₁ and In₂ are substituted by one or more substituents, the substituents are selected from the group consisting of a halogen atom, C₁ to C₁₀ alkyl, C₅ to C₁₅ aryl, C₆ to C₂₅ alkylaryl, and N- or P-containing alkyl or aryl. Exemplary metallocene compounds of this type include, but are not limited to, g-dimethylsilylbis(indenyl)hafniumdimethyl and μ-dimethylsilylbis(indenyl)zirconiumdimethyl.

In other embodiments, the metallocene compound may be a bridged bisindenyl metallocene having the general formula (In¹)Y(In²)MX₂, where In¹ and In² are identical 2,4-substituted indenyl groups bound to M and bridged by Y, Y is a bridging group in which the number of atoms in the direct chain connecting In¹ with In² is from 1 to 8 and the direct chain comprises C or Si, and M is a Group 3, 4, 5, or 6 transition metal. In¹ and In² are substituted in the 2 position by a methyl group and in the 4 position by a substituent selected from the group consisting of C₅ to C₁₅ aryl, C₆ to C₂₅ alkylaryl, and N- or P-containing alkyl or aryl. Exemplary metallocene compounds of this type include, but are not limited to, (μ-dimethylsilyl)bis(2-methyl-4-(3′,5′-di-tert-butylphenyl)indenyl)zirconiumdimethyl, (μ-dimethylsilyl)bis(2-methyl-4-(3′,5′-di-tert-butylphenyl)indenyl)hafniumdimethyl, (μ-dimethylsilyl)bis(2-methyl-4-naphthylindenyl)zirconiumdimethyl, (μ-dimethylsilyl)bis(2-methyl-4-naphthylindenyl)hafniumdimethyl, (μ-dimethylsilyl)bis(2-methyl-4-(N-carbazyl)indenyl)zirconiumdimethyl, and (μ-dimethylsilyl)bis(2-methyl-4-(N-carbazyl)indenyl)hafniumdimethyl.

Alternatively, in one or more embodiments of the present invention, the metallocene compound may correspond to one or more of the formulas disclosed in U.S. Pat. No. 7,601,666. Such metallocene compounds include, but are not limited to, dimethylsilyl bis(2-(methyl)-5,5,8,8-tetramethyl-5,6,7,8-tetrahydrobenz(f)indenyl)hafnium dimethyl, diphenylsilyl bis(2-(methyl)-5,5,8,8-tetramethyl-5,6,7,8-tetrahydrobenz(f)indenyl)hafnium dimethyl, diphenylsilyl bis(5,5,8,8-tetramethyl-5,6,7,8-tetrahydrobenz(f)indenyl)hafnium dimethyl, diphenylsilyl bis(2-(methyl)-5,5,8,8-tetramethyl-5,6,7,8-tetrahydrobenz(f) indenyl)zirconium dichloride, and cyclo-propylsilyl bis(2-(methyl)-5,5,8,8-tetramethyl-5,6,7,8-tetrahydrobenz(f) indenyl)hafnium dimethyl.

In one or more embodiments of the present invention, the activators of the catalyst systems used to produce propylene-based polymers comprise a cationic component. In some embodiments, the cationic component has the formula [R¹R²R³AH]⁺, where A is nitrogen, R¹ and R² are together a —(CH₂)ₐ— group, where a is 3, 4, 5 or 6 and form, together with the nitrogen atom, a 4-, 5-, 6- or 7-membered non-aromatic ring to which, via adjacent ring carbon atoms, optionally one or more aromatic or heteroaromatic rings may be fused, and R³ is C₁, C₂, C₃, C₄ or C₅ alkyl, or N-methylpyrrolidinium or N-methylpiperidinium. In other embodiments, the cationic component has the formula [RₙAH]⁺, where A is nitrogen, n is 2 or 3, and all R are identical and are C₁ to C₃ alkyl groups, such as for example trimethylammonium, trimethylanilinium, triethylammonium, dimethylanilinium, or dimethylammonium.

In one or more embodiments of the present invention, the activators of the catalyst systems used to produce the propylene-based polymers comprise an anionic component, [Y]⁻. In some embodiments, the anionic component is a non-coordinating anion (NCA), having the formula [B(R⁴)₄]⁻, where R⁴ is an aryl group or a substituted aryl group, of which the one or more substituents are identical or different and are selected from the group consisting of alkyl, aryl, a halogen atom, halogenated aryl, and haloalkylaryl groups. In one or more embodiments, the substituents are perhalogenated aryl groups, or perfluorinated aryl groups, including but not limited to perfluorophenyl, perfluoronaphthyl and perfluorobiphenyl.

Together, the cationic and anionic components of the catalysts systems described herein form an activator compound. In one or more embodiments of the present invention, the activator may be N,N-dimethylanilinium-tetra(perfluorophenyl)borate, N,N-dimethylanilinium-tetra(perfluoronaphthyl)borate, N,N-dimethylanilinium-tetrakis(perfluorobiphenyl)borate, N,N-dimethylanilinium-tetrakis(3,5-bis(trifluoromethyl)phenyl)borate, triphenylcarbenium-tetra(perfluorophenyl)borate, triphenylcarbenium-tetra(perfluoronaphthyl)borate, triphenylcarbenium-tetrakis(perfluorobiphenyl)borate, or triphenylcarbenium-tetrakis(3,5-bis(trifluoromethyl)phenyl)borate.

Any catalyst system resulting from any combination of a metallocene compound, a cationic activator component, and an anionic activator component mentioned in the preceding paragraphs shall be considered to be explicitly disclosed herein and may be used in accordance with the present invention in the polymerization of one or more olefin monomers. Also, combinations of two different activators can be used with the same or different metallocene(s).

Suitable activators for the processes of the present invention also include alominoxanes (or alumoxanes) and aluminum alkyls. Without being bound by theory, an alumoxane is typically believed to be an oligomeric aluminum compound represented by the general formula (R^{x}—Al—O)ₙ, which is a cyclic compound, or R^{x} (R^{x}—Al—O)ₙAlR^{x}₂, which is a linear compound. Most commonly, alumoxane is believed to be a mixture of the cyclic and linear compounds. In the general alumoxane formula, R^{x} is independently a C₁-C₂₀ alkyl radical, for example, methyl, ethyl, propyl, butyl, pentyl, isomers thereof, and the like, and n is an integer from 1-50. In one or more embodiments, R^{x} is methyl and n is at least 4. Methyl alumoxane (MAO), as well as modified MAO containing some higher alkyl groups to improve solubility, ethyl alumoxane, iso-butyl alumoxane, and the like are useful for the processes disclosed herein.

Further, the catalyst systems suitable for use in the present invention may contain, in addition to the transition metal compound and the activator described above, additional activators (co-activators) and/or scavengers. A co-activator is a compound capable of reacting with the transition metal complex, such that when used in combination with an activator, an active catalyst is formed. Co-activators include alumoxanes and aluminum alkyls.

In some embodiments of the invention, scavengers may be used to “clean” the reaction of any poisons that would otherwise react with the catalyst and deactivate it. Typical aluminum or boron alkyl components useful as scavengers are represented by the general formula R^{x}JZ₂ where J is aluminum or boron, R^{x} is a C₁-C₂₀ alkyl radical, for example, methyl, ethyl, propyl, butyl, pentyl, and isomers thereof, and each Z is independently R^{x} or a different univalent anionic ligand such as halogen (Cl, Br, I), alkoxide (OR^{x}) and the like. Exemplary aluminum alkyls include triethylaluminum, diethylaluminum chloride, ethylaluminum dichloride, tri-iso-butylaluminum, tri-n-octylaluminum, tri-n-hexylaluminum, trimethylaluminum and combinations thereof. Exemplary boron alkyls include triethylboron. Scavenging compounds may also be alumoxanes and modified alumoxanes including methylalumoxane and modified methylalumoxane.

In some embodiments, the catalyst system used to produce the propylene-based polymers comprises a transition metal component which is a bridged bisindenyl metallocene having the general formula (In¹)Y(In²)MX₂, where In¹ and In² are identical substituted or unsubstituted indenyl groups bound to M and bridged by Y, Y is a bridging group in which the number of atoms in the direct chain connecting In¹ with In² is from 1 to 8 and the direct chain comprises C or Si, and M is a Group 3, 4, 5, or 6 transition metal. In¹ and In² may be substituted or unsubstituted. If In₁ and In₂ are substituted by one or more substituents, the substituents are selected from the group consisting of a halogen atom, C₁ to C₁₀ alkyl, C₅ to C₁₅ aryl, C₆ to C₂₅ alkylaryl, and N- or P-containing alkyl or aryl. In one or more embodiments, the transition metal component used to produce the propylene-based polymers is μ-dimethylsilylbis(indenyl)hafniumdimethyl.

### Meltblown Nonwoven Fabrics

The present invention is directed to meltblown nonwoven fabrics or fabrics comprising at least one elastic layer, wherein the elastic layer comprises a propylene-based polymer as described previously. In some embodiments, the nonwoven fabrics additionally comprise one or more facing layers (e.g., a spunbond layer) positioned on one or both sides of the elastic layer(s). As used herein, “nonwoven” refers to a textile material that has been produced by methods other than weaving. In nonwoven fabrics, the fibers are processed directly into a planar sheet-like fabric structure and then are either bonded chemically, thermally, or interlocked mechanically (or both) to achieve a cohesive fabric. As used herein, a “meltblown nonwoven fabric” is a fabric that includes at least one meltblown component (e.g., a meltblown layer such as a meltblown elastic layer); the term does not require that the entire fabric or fabric construction be meltblown.

The present invention is directed not only to nonwoven fabrics, but also to processes for forming nonwoven fabrics comprising the polymers described herein. In one or more embodiments, such methods comprise the steps of forming a molten polymer composition comprising a propylene-based polymer, forming fibers comprising the propylene-based polymer, and forming an elastic nonwoven layer from the fibers. In further embodiments, the process may further comprise the steps of forming one or more nonwoven facing layers, and disposing the elastic layer or layers upon the facing layer. Optionally, one or more facing layers may additionally be disposed upon the elastic layer or layers, such that the elastic layers are sandwiched between the facing layers.

The nonwoven fabrics of the present invention can be formed by any method known in the art. For example, the nonwoven fabrics may be produced by a meltblown or spunbond process. In certain embodiments herein, the elastic layer or layers of the fabrics of the invention are produced by a meltblown process. When the fabrics further comprise one or more facing layers, the facing layers may also be produced by a meltblown process, by a spunbond or spunlace process, or by any other suitable nonwoven process.

In one or more embodiments, the elastic layer or layers of the nonwoven fabrics described herein are composed primarily of a propylene-based polymer as described previously. While the elastic layers may comprise additives and/or fillers in addition to the propylene-based polymer and may be formed from a reactor blend comprising the propylene-based polymer, no additional homopolymers or copolymers of propylene are blended with the propylene-based polymers post-reactor to form the elastic layers. In other words, the elastic layers of the nonwoven fabrics are substantially free of other propylene homopolymers and copolymers added post-reactor. This results in improved meltblown nonwoven fabrics, because the post-reactor addition of other polymers, such as propylene homopolymers, increases the manufacturing cost and reduces the elastic performance of the fabrics.

As used herein, “meltblown fibers” and “meltblown fabrics” refer to fibers formed by extruding a molten thermoplastic material at a certain processing temperature through a plurality of fine, usually circular, die capillaries as molten threads or filaments into high velocity, usually hot, gas streams which attenuate the filaments of molten thermoplastic material to reduce their diameter, which may be to microfiber diameter. Thereafter, the meltblown fibers are carried by the high velocity gas stream and are deposited on a collecting surface to form a web or nonwoven fabric of randomly dispersed meltblown fibers. Such a process is generally described in, for example, U.S. Pat. Nos. 3,849,241 and 6,268,203. Meltblown fibers are microfibers that are either continuous or discontinuous, and, depending on the resin, may be smaller than about 10 microns (for example, for high MFR isotactic polypropylene resins such as PP3746G or Achieve™ 6936G1, available from ExxonMobil Chemical Company); whereas for certain resins (for example, Vistamaxx™ propylene-based elastomer, available from ExxonMobil Chemical Company) or certain high throughput processes such as those described herein, meltblown fibers may have diameters greater than 10 microns, such as from about 10 to about 30 microns, or about 10 to about 15 microns. The term meltblowing as used herein is meant to encompass the meltspray process.

Commercial meltblown processes utilize extrusion systems having a relatively high throughput, in excess of 0.3 grams per hole per minute (“ghm”), or in excess of 0.4 ghm, or in excess of 0.5 ghm, or in excess of 0.6 ghm, or in excess of 0.7 ghm. The fabrics of the present invention may be produced using commercial meltblown processes, or in test or pilot scale processes. In one or more embodiments of the present invention, the fibers used to form the nonwoven fabrics are formed using an extrusion system having a throughput rate of from about 0.01 to about 3.0 ghm, or from about 0.1 to about 2.0 ghm, or from about 0.3 to about 1.0 ghm. In some embodiments, the fibers used to form the nonwoven fabrics are formed with an extrusion system producing a die pressure less than or equal to about 3000 psi (20684 kPa), or less than or equal to about 2500 psi (17237 kPa), or less than or equal to about 2000 psi (13790 kPa), or less than or equal to about 1750 psi (12066 kPa), or less than or equal to about 1500 psi (10342 kPa). In the same or other embodiments, the die pressure of the extrusion system may be greater than 100 psi (689 kPa), or greater than 250 psi (1724 kPa), or greater than 500 psi (3447 kPa), or greater than 750 psi (5171 kPa), or greater than 1000 psi (6895 kPa).

In a typical spunbond process, polymer is supplied to a heated extruder to melt and homogenize the polymers. The extruder supplies melted polymer to a spinneret where the polymer is fiberized as passed through fine openings arranged in one or more rows in the spinneret, forming a curtain of filaments. The filaments are usually quenched with air at a low temperature, drawn, usually pneumatically, and deposited on a moving mat, belt or “forming wire” to form the nonwoven fabric. See, for example, in U.S. Pat. Nos. 4,340,563; 3,692,618; 3,802,817; 3,338,992; 3,341,394; 3,502,763; and U.S. Pat. No. 3,542,615. The term spunbond as used herein is meant to include spunlace processes, in which the filaments are entangled to form a web using high-speed jets of water (known as “hydroentanglement”).

The fibers produced in the spunbond process are usually in the range of from about 10 to about 50 microns in diameter, depending on process conditions and the desired end use for the fabrics to be produced from such fibers. For example, increasing the polymer molecular weight or decreasing the processing temperature results in larger diameter fibers. Changes in the quench air temperature and pneumatic draw pressure also have an affect on fiber diameter.

The fabrics described herein may be a single layer, or may be multilayer laminates. One application is to make a laminate (or “composite”) from meltblown fabric (“M”) and spunbond fabric (“S”), which combines the advantages of strength from spunbonded fabric and greater barrier properties of the meltblown fabric. A typical laminate or composite has three or more layers, a meltblown layer(s) sandwiched between two or more spunbonded layers, or “SMS” fabric composites. Examples of other combinations are SSMMSS, SMMS, and SMMSS composites. Composites can also be made of the meltblown fabrics of the invention with other materials, either synthetic or natural, to produce useful articles.

In certain embodiments, the meltblown nonwoven fabrics of the invention comprise one or more elastic layers comprising a propylene-based polymer as previously described and further comprise one or more facing layers positioned on one or both sides of the elastic layer(s). The facing layer or layers may comprise any material known in the art to be suitable for use in such layers. Examples of suitable facing layer materials include, but are not limited to, any available material typically used as a facing layer, such as polypropylene (PP), polyethylene (PE), polyethylene terephthalate (PET), polylactic acid (PLA), and polymer or fiber blends of two or more of the foregoing.

A variety of additives may be incorporated into the polymers used to make the fibers and fabrics described herein, depending upon the intended purpose. Such additives may include, but are not limited to, stabilizers, antioxidants, fillers, colorants, nucleating agents, dispersing agents, mold release agents, slip agents, fire retardants, plasticizers, pigments, vulcanizing or curative agents, vulcanizing or curative accelerators, cure retarders, processing aids, tackifying resins, and the like. Other additives may include fillers and/or reinforcing materials, such as carbon black, clay, talc, calcium carbonate, mica, silica, silicate, combinations thereof, and the like. Primary and secondary antioxidants include, for example, hindered phenols, hindered amines, and phosphates. Nucleating agents include, for example, sodium benzoate and talc. Also, to improve crystallization rates, other nucleating agents may also be employed such as Ziegler-Natta olefin products or other highly crystalline polymers. Other additives such as dispersing agents, for example, Acrowax C, can also be included. Slip agents include, for example, oleamide and erucamide. Catalyst deactivators are also commonly used, for example, calcium stearate, hydrotalcite, and calcium oxide, and/or other acid neutralizers known in the art.

The nonwoven products described above may be used in many articles such as hygiene products including, but not limited to, diapers, feminine care products, and adult incontinent products. The nonwoven products may also be used in medical products such as sterile wrap, isolation gowns, operating room gowns, surgical gowns, surgical drapes, first aid dressings, and other disposable items.

### EXAMPLES

With reference to the following examples and figures, the following identifiers are used:

Polymer A is a propylene-ethylene copolymer as described above, with an ethylene content of about 15 wt % and an MFR of about 18.5 g/10 min.

Three polymers, identified as Examples 1-3 herein, were prepared by visbreaking Polymer A with an organic peroxide (2,5-dimethyl-2,5-di(t-butylperoxy)hexane, available commercially as Luperox 101 from Arkema, Inc.) to produce higher MFR polymers. The polymers were made by extruding Polymer A along with the peroxide in a compounding extruder. In Example 1, Polymer A was visbroken with 330 ppm of peroxide to produce a propylene-based polymer having a final MFR of 32 g/10 min (ASTM D-1238, 2.16 kg @ 230° C.). In Example 2, Polymer A was visbroken with 700 ppm of peroxide to produce a propylene-based polymer having a final MFR of 51 g/10 min (ASTM D-1238, 2.16 kg @ 230° C.). In Example 3, Polymer A was visbroken with 1500 ppm of peroxide to produce a propylene-based polymer having a final MFR of 96 g/10 min (ASTM D-1238, 2.16 kg @ 230° C.). A comparative example, Example 4, was prepared by extruding Polymer A through the compounding extruder at the same conditions as Examples 1-3, but without adding peroxide, resulting in an unchanged MFR of 18.5 g/10 min (ASTM D-1238, 2.16 kg @ 230° C.). Additionally, 1000 ppm of an antioxidant (Irgastab FS 301, available from BASF Inc.) was added to each of Examples 1 through 4 to improve polymer stability during extrusion.

The polymers of Examples 1-4 were then meltblown using a BIAX Fiberfilm pilot line with a 25″ die and having 75 holes/inch (0.020″ diameter capillaries). Laminates were made by meltblowing the polymers to form an elastic layer directly onto a Jacob-Holm 50/50 PP/PET 30 gsm facing layer, and contacting with a second identical facing layer while the polymers were still hot to produce 3-layer laminates having the structure facing layer/elastic layer/facing layer.

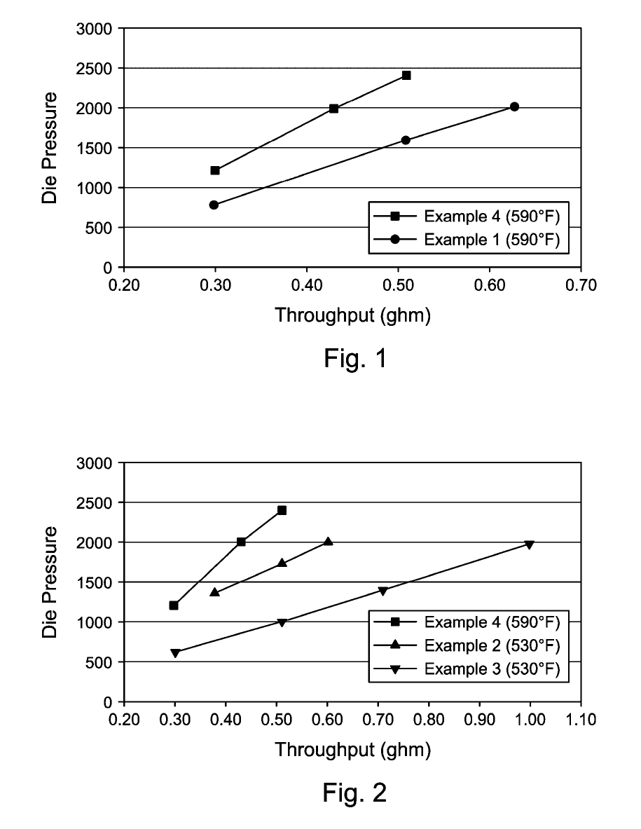

FIG. 1 depicts die pressure versus throughput rate for Example 1 and Comparative Example 4 at a melt temperature of 590° F. (310° C.) for both polymers. As shown in FIG. 1, the higher MFR polymer of Example 1 was able to be run at a higher throughput rate (0.63 grams/hole/min, or “ghm”) than the polymer of Example 4 (0.43 ghm) at a die pressure of 2000 psi (13790 kPa) and at the same melt temperature. At a throughput rate of 0.50 ghm, Comparative Example 4 generated a die pressure of 2400 psi (16547 kPa), which is above the desired maximum pressure of 2000 psi (13790 kPa). In contrast, however, Example 1 generated a pressure of only 1600 psi (11032 kPa) at the same throughput rate, a 33% decrease and well below the desired pressure limit of the die. Obtaining higher throughput rates at lower die pressures is desirable because it may allow higher production rates and improved profitability.

FIG. 2 depicts die pressure versus throughput rate for Examples 2 and 3 and Comparative Example 4, at a melt temperature of 530° F. (277° C.) for Examples 2 and 3 and a melt temperature of 590° F. (310° C.) for Example 4. As shown in FIG. 2, the melt temperature for the polymers of Examples 2 and 3 could be lowered by about 60° F. (15° C.) compared to that of Example 4 without having to lower the throughput rate. In fact, the higher viscosity of the polymer of Example 2 allowed it to be run at 0.60 ghm at 530° F. (277° C.) and 2000 psi (13790 kPa), or about 40% more than the rate of Comparative Example 4 at the same pressure and a higher melt temperature (590° F. (310° C.)). Further improvements were observed with the polymer of Example 3, which had the highest MFR of the polymers tested. Example 3 was run at more than twice the throughput rate of Example 4 without exceeding the pressure limit of the die (1.0 ghm at 530° F. (277° C.) and 1380 psi (9515 kPa) for Ex. 3 compared to 0.43 ghm at 590° F. (310° C.) and 2000 psi (13790 kPa) for Ex. 4)). The ability to run at a lower melt temperature widens the processing window, providing greater flexibility for optimizing parameters such as mechanical or aesthetic properties of the fabrics and reducing the thermal degradation rate of the polymers used to form the fabrics.

For purposes of convenience, various specific test procedures are identified above for determining certain properties. However, when a person of ordinary skill reads this patent and wishes to determine whether a composition or polymer has a particular property identified in a claim, then any published or well-recognized method or test procedure can be followed to determine that property, although the specifically identified procedure is preferred. Each claim should be construed to cover the results of any of such procedures, even to the extent different procedures can yield different results or measurements. Thus, a person of ordinary skill in the art is to expect experimental variations in measured properties that are reflected in the claims.

Having described the various aspects of the compositions herein, further specific embodiments of the invention include those set forth in the following lettered paragraphs:
A. A meltblown nonwoven composition having at least one elastic layer, wherein the elastic layer comprises a propylene-based polymer, the propylene-based polymer having from about 75 to about 95 wt % propylene and from about 5 to about 25 wt % ethylene and/or a C₄-C₁₂ alpha-olefin, and further having a triad tacticity greater than about 90%, a heat of fusion less than about 75 J/g, and an MFR greater than about 25 g/10 min (230° C., 2.16 kg).
B. The composition of paragraph A, wherein the propylene-based polymer comprises from about 8 to about 20 wt % ethylene.
C. The composition of any of paragraphs A-B, wherein the propylene-based polymer comprises from about 12 to about 18 wt % ethylene.
D. The composition of any of paragraphs A-C, wherein the propylene-based polymer has been treated with a visbreaking agent such that the melt flow rate of the propylene-based polymer after treatment with the visbreaking agent is at least 1.5 times the initial melt flow rate of the polymer prior to treatment.
E. The composition of paragraph D, wherein the propylene-based polymer has been treated with from about 100 to about 5000 ppm of the visbreaking agent.
F. The composition of any of paragraphs D-E, wherein the melt flow rate of the propylene-based polymer after treatment with the visbreaking agent is at least 3 times the initial melt flow rate of the polymer prior to treatment.
G. The composition of any of paragraphs D-F, wherein the visbreaking agent comprises an organic peroxide.
H. The composition of any of paragraphs D-G, wherein the organic peroxide is 2,5-dimethyl-2,5-di(t-butylperoxy)hexane.
I. The composition of any of paragraphs A-H, further comprising one or more facing layers, preferably where the elastic layer is a meltblown layer and the facing layer is at least one of a spunbond layer and a carded layer, and also preferably where the facing layer comprises polypropylene, polyethylene, or a combination thereof.
J. The composition of paragraph I, wherein the facing layer comprises polypropylene, polyethylene terephthalate, or a combination thereof
K. The composition of any of paragraphs A-J, wherein the elastic layer is substantially free of homopolymers or copolymers of propylene added post-reactor.
L. An article comprising the composition of any of paragraphs A-K.
M. The article of paragraph L, wherein the article is a diaper, adult incontinence product, or other personal hygiene product.
N. A process for producing a meltblown nonwoven fabric comprising forming a molten propylene-based polymer comprising from about 75 to about 95 wt % propylene and from about 5 to about 25 wt % ethylene and/or a C₄-C₁₂ alpha-olefin, and having a triad tacticity greater than about 90%, a heat of fusion less than about 75 J/g, and an MFR greater than about 25 g/10 min (230° C., 2.16 kg); forming fibers comprising the propylene-based polymer; and forming an elastic nonwoven fabric layer from the fibers.
O. The process of paragraph N, wherein the propylene-based polymer comprises from about 8 to about 20 wt %, or from about 12 to about 18 wt % ethylene.
P. The process of any of paragraphs N-O, further comprising the step of processing the molten propylene-based polymer with from about 100 to about 5000 ppm of a visbreaking agent comprising one or more organic peroxides until the polymer has been visbroken such that the melt flow rate of the polymer is at least about 1.5 times the initial melt flow rate of the polymer, prior to forming fibers comprising the visbroken propylene-based polymer.
Q. The process of paragraph P, wherein the molten propylene-based polymer is processed with from about 250 to about 2500 ppm of the visbreaking agent.
R. The process of any of paragraphs P-Q, wherein the melt flow rate of the propylene-based polymer after processing with the visbreaking agent is at least 3 times the initial melt flow rate of the polymer prior to processing.
S. The process of any of paragraphs P-R, wherein the visbreaking agent comprises 2,5-dimethyl-2,5-di(t-butylperoxy)hexane.
T. The process of any of paragraphs P—S, further comprising forming one or more nonwoven facing layers, and disposing the elastic nonwoven fabric layer upon the facing layer.
U. The process of paragraph T, wherein the facing layer comprises polypropylene, polyethylene terephthalate, or a combination thereof.
V. The process of any of paragraphs P-U, wherein the fibers are formed using an extruder having a throughput rate of from about 0.1 to about 2.0 ghm.
W. The process of any of paragraphs P-V, wherein the fibers are formed using an extruder having a die pressure less than or equal to about 3000 psi (20684 kPa).
X. The process of any of paragraphs P-W, wherein the fibers are formed using an extruder having a die pressure less than or equal to about 3000 psi (20684 kPa) and greater than 500 psi (3447 kPa).
Y. The process of any of paragraphs P-X, wherein the elastic nonwoven fabric layer is substantially free of other propylene-based homopolymers or copolymers added post-reactor.
Z. An article comprising an elastic nonwoven fabric layer formed by the process of any of paragraphs P-Y.
AA. The composition according to any of paragraphs A-K, wherein the composition and/or the elastic layer further comprises at least one of carbon black, clay, talc, calcium carbonate, mica, silica, and silicate.
AB. The composition according to any of paragraphs A-K, wherein the composition and/or the elastic layer further comprises at least one of carbon black, clay, talc, calcium carbonate, mica, silica, silicate, hindered phenol, hindered amine, phosphate, sodium benzoate, oleamide, erucamide, calcium stearate, hydrotalcite, and calcium oxide.

Certain embodiments and features have been described using a set of numerical upper limits and a set of numerical lower limits. It should be appreciated that ranges from any lower limit to any upper limit are contemplated unless otherwise indicated. Certain lower limits, upper limits and ranges appear in one or more claims below. All numerical values are “about” or “approximately” the indicated value, and take into account experimental error and variations that would be expected by a person having ordinary skill in the art.

As used herein, the phrases “substantially no,” “substantially free of,” and “substantially free of” are intended to mean that the subject item is not intentionally used or added in any amount, but may be present in very small amounts existing as impurities resulting from environmental or process conditions.

To the extent a term used in a claim is not defined above, it should be given the broadest definition persons in the pertinent art have given that term as reflected in at least one printed publication or issued patent. Furthermore, all patents, test procedures, and other documents cited in this application are fully incorporated by reference to the extent such disclosure is not inconsistent with this application and for all jurisdictions in which such incorporation is permitted.

While the foregoing is directed to embodiments of the present invention, other and further embodiments of the invention may be devised without departing from the basic scope thereof, and the scope thereof is determined by the claims that follow.

What we claim:

1. A meltblown nonwoven composition having at least one elastic layer, wherein the elastic layer comprises a propylene-based polymer, the propylene-based polymer having:
a. from about 75 to about 95 wt % propylene and from about 5 to about 25 wt % ethylene and/or a C₄-C₁₂ alpha-olefin;
b. a triad tacticity greater than about 90%;
c. a heat of fusion less than about 75 J/g; and
d. an MFR greater than about 25 g/10 min (230° C., 2.16 kg),
wherein the propylene-based polymer has been treated with a visbreaking agent such that the melt flow rate of the propylene-based polymer after treatment with the visbreaking agent is at least 1.5 times the initial melt flow rate of the polymer prior to treatment.

2. The composition of claim 1, wherein the propylene-based polymer comprises from about 8 to about 20 wt % ethylene.

3. The composition of claim 2, wherein the propylene-based polymer comprises from about 12 to about 18 wt % ethylene.

4. The composition of claim 1, wherein the elastic layer is substantially free of homopolymers or copolymers of propylene added post-reactor.

5. The composition of claim 1, wherein the propylene-based polymer has been treated with from about 100 to about 5000 ppm of the visbreaking agent.

6. The composition of claim 1, wherein the melt flow rate of the propylene-based polymer after treatment with the visbreaking agent is at least 3 times the initial melt flow rate of the polymer prior to treatment.

7. The composition of claim 1, wherein the visbreaking agent comprises an organic peroxide.

8. The composition of claim 7, wherein the organic peroxide is 2,5-dimethyl-2,5-di(t-butylperoxy)hexane.

9. The composition of claim 1, wherein the composition and/or the elastic layer comprises at least one of carbon black, clay, talc, calcium carbonate, mica, silica, silicate, hindered phenol, hindered amine, phosphate, sodium benzoate, oleamide, erucamide, calcium stearate, hydrotalcite, and calcium oxide.

10. The composition of claim 1, wherein the elastic layer further comprises at least one of carbon black, clay, talc, calcium carbonate, mica, silica, and silicate.

11. The composition of claim 1, further comprising one or more facing layers.

12. The composition of claim 11, wherein the facing layer comprises polypropylene, polyethylene terephthalate, or a combination thereof.

13. The composition of claim 12, wherein the elastic layer is a meltblown layer and the facing layer is at least one of a spunbond layer and a carded layer.

14. The composition of claim 13, wherein the facing layer comprises polypropylene, polyethylene, or a combination thereof.

15. An article comprising the composition of claim 1.

16. The article of claim 15, wherein the article is a diaper, adult incontinence product, or other personal hygiene product.
